(19) United States
(12) Patent Application Publication
(10) Pub. No.: US 2025/0381408 A1
(43) Pub. Date: Dec. 18, 2025

(54) ATRIAL PACING CAPTURE CONFIRMATION STRATEGY IN AN INTRA-CARDIAC LEADLESS PACEMAKER

(71) Applicant: BIOTRONIK SE & CO. KG, Berlin (DE)

(72) Inventors: Madeline Anne MIDGETT, Portland, OR (US); Brian M. TAFF, Portland, OR (US); R. Hollis WHITTINGTON, Portland, OR (US); Kurt SWENSON, Corvallis, OR (US); Hannes KRAETSCHMER, West Linn, OR (US)

(73) Assignee: BIOTRONIK SE & CO. KG, Berlin (DE)

(21) Appl. No.: 18/879,889

(22) PCT Filed: Jun. 20, 2023
(86) PCT No.: PCT/EP2023/066655

§ 371 Date: Dec. 30, 2024

Related U.S. Application Data
(60) Provisional application No. 63/388,755, filed on Jul. 13, 2022.

(30) Foreign Application Priority Data
Jul. 29, 2022 (EP) 22187880.4

(51) Int. Cl. A61N 1/37; A61N 1/375

(52) U.S. Cl. CPC A61N 1/3714; A61N 1/37518; A61N 1/3756

(57) ABSTRACT

A leadless pacing device for implanting into an atrium of a heart. The leadless device comprises an implant anchor for connecting the device to an inner wall of the atrium; a stimulator for a direct stimulation of the atrium; a sensor for sensing a ventricular activity of the heart corresponding to the direct stimulation of the atrium. Further aspects relate to a system comprising such leadless pacing device, a method and a computer program that may be executed by such leadless pacing device.

### CROSS-REFERENCE TO RELATED APPLICATIONS

This application is the United States National Phase under 35 U.S.C. § 371 of PCT International Patent Application No. PCT/EP2023/066655, filed on Jun. 20, 2023, which claims the benefit of European Patent Application No. 22187880.4, filed on Jul. 29, 2022 and U.S. Provisional Patent Application No. 63/388,755, filed on Jul. 13, 2022, the disclosures of which are hereby incorporated by reference herein in their entireties.

### TECHNICAL FIELD

The present invention generally relates to a leadless pacing device, (e.g., a leadless pacemaker), that may be implantable into an atrium of a heart and a system, a method, and a computer program for operating such device, particularly for sensing a ventricular activity of the heart corresponding to a direct stimulation of the atrium.

### BACKGROUND

Devices that can be implanted into a patient for sensing activity of the heart of the patient and/or to pace or defibrillate the heart have long been known. Traditionally, lead-based cardiac devices have been used which may have their main unit outside of the heart (e.g., in a chest area), wherein the inner walls of the heart chambers may be contacted by the ends of a transvenous lead system extending from the main unit. More recently, such implants have been provided or presented as leadless devices. A leadless implant may be directly implanted into a heart in a self-contained manner, (e.g., it does not comprise external, such as transvenous, leads for interacting with the heart). So far, leadless implants for implantation into the right ventricle have been used. They may facilitate cardiac therapies over their direct contact to the ventricle as a self-contained system.

A known leadless device may comprise a stimulator and a sensor in direct contact with the inner wall of the right ventricle. This approach enables direct electrical stimulation (e.g., pacing) of the right ventricle and direct sensing of electrical signals corresponding to a paced ventricular event. Said leadless device thus enables therapies based on ventricular pacing and ventricular sensing, wherein a specific stimulation may be based on a specific ventricular activity (e.g., VVI mode).

It would be desirable to expand leadless device therapies also to atrially-stationed pacing device. Due to size restrictions of an atrium, it is desirable to design such devices with low power consumption, as the available battery size may be even more limited than for devices in the ventricle.

In the realm of leaded pacemakers, devices have been known that use atrial capture control to minimize power consumption. For example, it is known to sense atrial events for that matter. However, this approach does not consider further reactions of the heart to the atrial stimulus, for example, the ventricular activity or the atrially-evoked response signal. These heart reactions may be of particular interest for bradycardia or sinus arrest therapies. The feature support power levels associated with these legacy offerings may not be optimal, particularly when considered for leadless systems stationed in an atrium and their affiliate constraints for on-board batter sizing.

Further, lead-based systems, may generally be disadvantaged compared to leadless systems, due to their direct physical pathway for infection (especially infections initiated within legacy device chest pockets) to access the heart. Moreover, they place volumes of hardware within the patient that can be difficult to explant and include components that are subject to mechanical failure in the harsh physiological environments where they reside. It is, in fact, these very complications (i.e., infection risk and lead failure) that have largely driven the emergence of leadless pacing.

Overall, there is therefore the tendency to replace lead-based devices by leadless devices which result in reduced stress on the patient, reduced infection risk and reduced risk of device failure.

Despite the above, the currently known techniques for leadless cardiac implants may not always be optimal, in particular when it comes to control of their power consumption driving a further need to improve leadless cardiac implants. Specifically, the strict power limitations specific to leadless pacemakers intended for an atrium create a need for innovative strategies that reduce the pacing output beyond standard pacer levels while maintaining safe and reliable therapy.

The present disclosure is directed toward overcoming one or more of the above-mentioned problems, though not necessarily limited to embodiments that do.

### SUMMARY

The aspects described herein address the above need at least in part.

A first aspect relates to a leadless pacing device for implanting into an atrium of a heart. It may comprise an implant anchor for connecting the device to an inner wall of the atrium, a stimulator for a direct stimulation of the atrium, and a sensor for sensing a ventricular activity of the heart corresponding to the direct stimulation of the atrium. The implant anchor may also be called a connector.

This is based on the idea that the device, fully residing within the atrium, may sense a ventricular response corresponding to a direct stimulation of the atrium. This concept may be of particular interest for cardiac therapies which may rely on atrial stimulations, particularly when these are intended to create ventricular responses (e.g., for patients with bradycardia and/or sinus arrest). As such, the aim of an atrial stimulation may be to cause a desired ventricular activity. For example, this may typically be a corresponding ventricular response (i.e., a ventricular contraction). However, there may also be cases where an atrial stimulus of a cardiac therapy is intended to cause an atrial response (e.g., an atrial contraction) which may then be conducted to the ventricle causing a ventricular response. In that regard, the atrial response may be determined by the corresponding ventricular activity. Overall, in some therapy applications of the leadless device, its atrial stimulus may be the input signal to the heart system, whereas the ventricular activity may be the desired output signal. The atrial response detected may be (also) another means for determining capture.

Hence, according to the above aspect, an optimized approach for determining the functionality of the leadless device stationed in the atrium may be provided, since it allows sensing the desired output, (i.e., the corresponding ventricular activity). This may in particular not only allow verifying the device's overall functionality (i.e., verifying that the desired output is achieved), but also optimizing power consumption, by setting the stimulation power to a (minimum) level, that still supports the intended ventricular response.

Prior art approaches based on leaded devices suffer from merely sensing atrial events which may not cover the intended therapy output to a full extent. Hence, in such scenarios, a fixed, high-output pace setting may be required that would lead to an excess energy consumption. In contrast, the present disclosure allows directly associating a sensed ventricular activity (i.e., a contraction) with a paced atrial event allowing for optimized functionality checks and power control.

The implant anchor may provide a stable connection to the wall of the atrium, such that a direct stimulation to the atrium may be provided as well as a reliable sensing of ventricular activity (e.g., via far-field sensing).

The implant anchor may be configured for fixedly mounting the leadless device to the inner wall of the atrium, by providing a direct mechanical connection with the inner wall. The stimulator may be in the vicinity of the implant anchor, such that the latter may ensure a direct electrical contact of the stimulator to the atrium. The implant anchor may be constructed as the base mount of the leadless device serving, e.g., as the stable surgical fix point and/or a bottom face of the leadless device when implanted into the atrium. The stimulator may also be formed at the base of the leadless device. The stimulator may thus be, for example, pressed directly against the inner wall of the atrium by means of the implant anchor, and may thus apply direct myocardial stimulation to the atrium.

The sensor may be adapted to sense the ventricular activity in an autarkic manner (i.e., without assistance from elements outside of the atrium). The sensor for sensing ventricular activity and the stimulator may, at least in part, share elements (e.g., they may use the same electrode) or the sensor and stimulator may be separate elements of the leadless device (e.g., have a separate electrode, each). The sensor and/or stimulator may be formed at the base of the leadless device. They (i.e., their electrode(s)) may be, for example, pressed directly against the inner wall of the atrium by means of the implant anchor, similarly as outlined herein with reference to the stimulator. For example, the sensor may in that regard sense the signal from the ventricular activity (far-field sensing).

In another example the device may be configured to determine an occurrence or an absence of a ventricular event corresponding to the direct stimulation of the atrium, based at least in part on the sensed ventricular activity. A ventricular event may be a significant or insignificant ventricular activity taking place (i.e., a measurable ventricular contraction or lack thereof). The device may be configured to determine a ventricular event based on the sensed ventricular activity. This may enable a precise classification of the ventricular activity as a particular ventricular event corresponding to the atrial stimulation.

The determination of a ventricular event may be achieved by determining the presence of a pulse signal in the sensed ventricular activity signal. For example, the determining may comprise a rising edge, falling edge and/or a certain threshold, of the ventricular activity signal. The correspondence with the direct atrial stimulation may, for example, be established based on the ventricular event occurring after the stimulation of the atrium, but before a timer lapses. Additionally or alternatively, it is also possible to compare signals shapes etc. to establish correspondence.

The determination of a ventricular event may also comprise the use of a detection algorithm which may consider at least one signal parameter of the corresponding ventricular activity (e.g., shape, amplitude, duration, frequency, etc.) to determine a particular ventricular event. For example, the detection algorithm may be configured to determine various types of ventricular events, which may relate to types of ventricular contractions (e.g., a present or an absent contraction, and/or types of present contractions such as. low/high amplitude conditions, short/long durations, particular shapes, etc.).

The determination of an occurrence or an absence of a determined ventricular event may be assessed in various ways (e.g., by Boolean logic). In an example, the device may test if a predetermined ventricular event (e.g., a particular type as outlined above) has been determined and may assign an attribute to the corresponding ventricular event to indicate its occurrence or absence. The device may be configured to precisely screen for one type of ventricular event (e.g., a present contraction) but may also be configured to screen for a plurality of types of ventricular events (e.g., a present contraction, with a predetermined duration, etc.). The signal processing of the detection algorithm may comprise various steps, for example, scaling, filtering, rectification, etc. In addition, the device may comprise a high-gain amplifier to optimally convert the originally sensed signal for determination of a ventricular event. The leadless device may further be configured for collecting high-resolution and/or high-gain intracardiac electrogram (IEGM) data for use in the detection algorithm. The leadless device may comprise hardware to support the implementation of various types of signal processing.

In another example, the device may be configured to determine whether a predetermined direct stimulation, at a predetermined stimulation power and/or a predetermined stimulation energy, leads to a corresponding ventricular event. For example, a direct stimulation at the predetermined stimulation power and/or energy may be applied. Subsequently, it may be determined, based on the sensed ventricular activity, whether a corresponding ventricular event occurs. The stimulation may be, for example, at least one pulse with a specific energy.

The pulse may have an effective power, which may be defined by the ratio of the pulse energy to its duration and/or its full width at half maximum (FWHM). In an example the predetermined stimulation may comprise various parameters (e.g., cycle length, current, voltage, pulse width, total power, average power, total energy, etc.) wherein the set of parameters may be related to the occurrence or absence of a corresponding ventricular event. The determined result may be stored by the device (e.g., in a table form, database, etc.). In a further example, the device may be configured to change a stimulation parameter of the predetermined direct stimulation. For example, the device may be configured to change a stimulation power and/or a stimulation energy of the direct stimulation. For example, the device may comprise a power electronic circuitry coupled to the stimulator to modulate the amount of stimulation power which may be applied onto the atrium. The device may be further configured to adjust various further parameters of the direct stimulation, (e.g., cycle length, current, voltage, pulse width, etc.). For example, a pulse with a fixed duration may be applied at different amplitudes, leading to different energies and powers. As a further example, a pulse with a fixed energy may be varied in duration, leading to different powers but identical energies.

In another example, the device may be configured to perform multiple or a plurality of direct stimulations with different stimulation powers and/or stimulation energies to determine a threshold of the stimulation power and/or of the stimulation energy for which a ventricular event occurs. In an example, the device may be configured to determine a capture threshold of the atrial stimulation. Capture may mean that a direct atrial stimulation has effectively resulted in the occurrence of a corresponding desired ventricular event (e.g., a present ventricular contraction). The threshold may mean a capture threshold of the stimulation power for which a desired corresponding ventricular event occurs. An atrial stimulation power below the threshold may result in the absence of the desired corresponding ventricular event. In this case, the heart activity may not be sufficiently stimulated by the stimulation power to evoke a corresponding response. It may be key to determine the threshold of the stimulation power and/or energy to minimize the power consumption which is highly critical in leadless pacemaker platforms. As a fully contained system stationed within the atrium, battery exchange may not be possible (at least not without surgery which may cause medical stress on the patient having the implant). Hence, a lower power consumption may lead to a significant increase in device longevity and may mitigate premature battery exchange by medical intervention.

For example, the device may, after determination of the threshold, apply subsequent stimulations at that threshold. The determination routine may then be carried out again at a later time (e.g., it may be carried out once per hour, once per day, etc.) to verify whether the threshold is still correct. To avoid pacing at a power that runs the risk of being too low, a safety margin may be added to the determined threshold, and then the device may operate accordingly. Also, the determination routine may be triggered in an event-based manner, (e.g., upon implantation and/or a follow-up).

For example, the device may be configured to adjust its atrial stimulation power according to the determined power threshold. This may be by way of a tracking mode, wherein the device performs a threshold search in regular time intervals to adjust its atrial stimulation power, which may be used for therapeutic stimulation (e.g., pacing). This ensures the functionality of the leadless pacing device while minimizing power consumption, avoiding unnecessarily high-power stimulations and may increase device longevity. In another example, the regular (e.g., periodic) threshold measurements may be used to monitor the power threshold for statistical purposes, which may be stored on the leadless pacing device for a later readout (e.g., by a clinician or otherwise).

In an example, the device may be configured to determine the minimum threshold of the stimulation power/energy which may lead to a present ventricular contraction by ways of various algorithm steps. The determination of the minimum threshold may also be referred to as a threshold search. The determination may be based on at least one occurrence and at least one absence of a corresponding ventricular event. As an example, the initial direct atrial stimulation may be based on a first predetermined power (the following algorithm may also be based on stimulation energy and/or pulse duration instead of stimulation power, although this will not be repeated for the sake of brevity), which may result in the occurrence of a corresponding ventricular event with a high probability. The predetermined power may be a high output power, which is known to cause a ventricular contraction. If the device determines the absence of a corresponding ventricular event at the high output power, the next stimulation may be based on an even higher output power (e.g., in one or more steps) up until the maximum power allowed by the device. If still no ventricular event can be determined, an alarm may be issued to an external device. If the device determines the occurrence of the corresponding ventricular event at the high output power (or at the even higher or possibly maximum output power), the next stimulation may be based on a power lower by a first increment than the high output power (or the even higher or possibly maximum output power). Further, the device may determine if the incrementally lower power results in the occurrence or absence of the corresponding ventricular event. If the occurrence was determined, the device may sequentially repeat the step with incrementally lower stimulation powers until the first absence of a corresponding ventricular event is determined at a first absence power. This may indicate that the respective stimulation power is in the vicinity of the threshold. In an example, the incrementally higher stimulation power prior to the first absence power may be defined as the minimum threshold of the stimulation power.

In another example, the first increment (i.e., the prior stimulation power difference) may be reduced to a second increment after the first absence is determined. This may enable a finer grid to determine a threshold power with subsequent stimulation steps, similarly, as outlined above. For example, the device may further apply an atrial stimulation with a power higher by the second increment than the first absence power and determine the occurrence or absence of the corresponding ventricular event. The step may be repeated until an occurrence of the corresponding event is determined, which may then be used as the minimum threshold. Various other power threshold search algorithms may be possible, which for brevity purposes are not discussed. For example, after finding the minimum threshold based on the second increment, a third increment may be used that is smaller than the second increment.

The stimulation power may again be lowered in steps corresponding to the third increment, until an absence of a corresponding ventricular event is determined, etc.

In an example, the device may be configured to sense the ventricular activity and/or to determine the ventricular event in a predetermined time window after the direct stimulation. This may optimize the device's capabilities to detect the corresponding ventricular activity since the detection is narrowed to a meaningful time window in which the corresponding ventricular activity may be expected. This may isolate interference signals and/or ventricular activity not related to the atrial stimulus. It may further significantly reduce the computing complexity of the signal evaluation of the ventricular activity as implemented by the detection algorithm since the ventricular signal is reduced to a relevant monitoring window. In addition, this may reduce power consumption by the device due to the reduction of computing complexity (which may require only computing steps for signal data inside the time window by the inventive concept). This approach of narrowing the signal detection to a time window may be used in the threshold search outlined herein. Thus, the possible multiple steps of the threshold search may require significantly less effort to determine an occurrence or absence of a ventricular event and may significantly reduce the power consumption of the leadless device.

In an example, the device may be configured to determine at least one parameter of the time window at least in part based on data, stored by the device, concerning at least one previously measured time interval of the heart. In an example the at least one parameter may comprise the beginning, center, duration and/or end of the time window. The parameters of the time window may be based on a time referenced to the atrial stimulation (e.g., the start of the atrial stimulation may be considered the initial time) wherein the time window parameters are referenced to said initial time. The data may be based on the patient's ApVs (atrial paced, ventricle sensed) interval history. The history may be based on at least one prior determined ApVs interval for therapeutic purposes and/or a test stimulation. The history may also be based on one or more parameters of a prior time window used for determining a corresponding ventricular activity and/or ventricular event, as outlined herein. The at least one parameter may be set to match at least one parameter of the last saved ApVs interval (e.g., the center of the time window). In an example, the time window duration may have a fixed value, which may consider cycle-to-cycle variation of ventricular events, wherein merely the center of the time window is determined based on the previous ApVs interval. In other examples, also the time window duration may be set based on stored data, taking into account patient-specific irregularities. The at least one parameter may also be set to an average of a (short) history of determined ApVs intervals pulled from a buffer history or a rolling window assessment.

In an example, the device may be configured to determine at least one parameter of the time window at least in part based on performing a test stimulation, with a test stimulation power and/or a test stimulation energy (or any other stimulation parameter as outlined herein), and sensing the corresponding ventricular activity. The underlying idea centers on causing the desired corresponding ventricular event by the test stimulation and quantitatively determining its time window parameters. As an example, this may take the form of an initialization phase, wherein a high-power atrial stimulation is applied (e.g., as part of the threshold determination routine outlined above), which is expected to cause the occurrence of a corresponding ventricular event/contraction. After the high-power atrial stimulation, the ventricular activity may be continuously sensed which may enable the device to eventually pick up the signal of the corresponding ventricular event. The device may be configured to determine the occurrence of the ventricular event out of the continuous ventricular signal, as well as further parameters of the ventricular event in reference to the time at which the high-power atrial stimulation took place. This approach may enable the determination of at least one time window parameter relevant to the time window in which the ventricular event occurs (e.g., the duration of the ventricular event and when it takes place after the atrial stimulation). As an example, the device may be configured to implement a respective ApVs detection algorithm to determine the time window (and/or at least one parameter of the time window) in which the corresponding ventricular event occurs. The device may be configured to subsequently determine a threshold of the stimulation power with various stimulation powers for which a ventricular event occurs, whereas the determination of the occurrence or absence may be narrowed to the recently determined time window, as outlined herein. Possibly, the window may be adjusted after a sensing step taking into account possible drifts, if, for example, the event is detected outside the center of the window.

This approach may initially require a higher signal processing complexity (e.g., needed for the ApVs detection algorithm) but may allow determining a suitable time window based on most recent measurements which may then not only be used to operate the device later on, but potentially also already for the thresholding routine. For (both) applications, this approach may greatly reduce the risk of using erroneous time windows that may lead to a false-negative result of the determination of a ventricular event.

It may be required to have stable conduction from the atrium to the ventricle (e.g., 1:1 AV conduction) during the power threshold determination. To achieve a stable ApVs rhythm during the test, the atrial stimulation rate (i.e., pacing rate) may be increased to overdrive the intrinsic activity of the heart and an AV pacing delay may be lengthened to encourage sensing. In a further example, a high-output, atrial stimulation may be applied after every stimulation part of the threshold determination (e.g., every stimulation step of the threshold search is followed by a high-output stimulation). This may maintain a stable ApVs during the further threshold determination steps.

In an example, the sensor may be configured for far-field sensing. Far-field sensing of ventricular activity by means of an atrially-stationed device may be advantageous, since the (relatively strong) ventricular signals (R-wave) may be determined in the atrium with still beneficial signal to noise ratio, as the disturbance by atrial signals (P-wave) is relatively weak. Hence, a ventricular event (e.g., contraction) may be reliably derived from signals of the sensor. For example, the sensor may detect the electrical signal of the far-field ventricular activity picked up at the atrium to which the sensor may be directly connected. The far-field sensing may require a specific signal processing implemented by the device. For example, this may require filtering the atrial signal out and determining if the signal originates from the ventricle.

It is noted that the sensor may be understood as (integral) part of the atrially-implanted device, such that it performs a remote sensing of ventricular activity.

In another example, the sensor may be based on the mechanical signal of the ventricular activity, wherein the leadless device may comprise an accelerometer (and/or any other motion detector) for sensing ventricular activity. The accelerometer may be configured to provide signals that allow for a sensing of the ventricular activity (from the atrium) by means of the mechanical signatures the ventricular activity generates in the atrium. Also in this way, reliable measurements of the relatively strong ventricular activity (or the corresponding mechanical signatures in the atrium) may be facilitated.

In an example, the sensor may additionally, or alternatively, be configured for receiving information on ventricular activity from at least one first additional sensor for implanting in a ventricle of the heart. The device may thus be configured to determine a ventricular activity corresponding to the atrial stimulation based on sensory data of the first additional sensor which may be implanted into the ventricle. The information on the ventricular activity may be based on directly sensed ventricular activity, e.g., from a directly picked-up signal from the inner wall of the ventricle.

A second aspect relates to a system which may comprise the leadless pacing device and the at least one first additional sensor. The devices in the system may be configured as outlined herein. In an example the leadless pacing device may be stationed in the right atrium, wherein the at least one first additional sensor may be stationed in the right ventricle. The system may further comprise a plurality of first additional sensors which may be stationed in the ventricles, wherein the leadless pacing device may also be stationed in the left atrium.

In an example, the first additional sensor may be a sensor stationed in the (right) ventricle comprising a passive component. For example, it may comprise a capacitor coupled to the ventricle (e.g., to the inner wall of the ventricle, or to ventricular muscle cells), wherein its dielectric component may be configured to be exposed to the electrical influence of the ventricle. Hence, an electrical excitation of the ventricle (e.g., of the ventricular muscle cells) may cause a change in the electrical field of the dielectric component of the capacitor, which may lead to a change in the capacitance of the capacitor. The first additional sensor may thus sense ventricular depolarizations (i.e., ventricular activity) by a change of said capacitance. The first additional sensor may comprise a resonant circuit which the capacitor may be part of or coupled to, which may provide the means for a readout of the capacitance change. For example, the capacitor may be coupled to an inductance (e.g., a coil structure) in the resonant circuit. This may form a resonant frequency which depends on the capacitance of the capacitor. The change in ventricular activity may be sensed by the change in the resonant frequency. An antenna may relay the sensed information (e.g., the change in resonant frequency) to the leadless device (in the atrium). For example, the information may be sent from the antenna to a receiver unit of the leadless device, whereas the device may be configured to apply further processing of said information to determine the ventricular activity. The antenna may be comprised in the resonant circuit and/or may be comprised in the leadless device. In an example, the antenna may comprise the inductance, with the inductance being connected to the capacitor.

Notably, the at least one first additional sensor that may be for implanting in a ventricle and that may be configured for sensing ventricular activity and sending information on ventricular activity to the leadless device for implanting in an atrium is also separate part of the present disclosure. The first additional sensor may have its own power source but may also be constructed to work without an internal power source.

In an example, the system may comprise one or more additional leadless devices. For example, the system may then be configured for sensing a ventricular activity of the heart corresponding to the direct stimulation of the atrium, based on the ventricular activity sensed in a plurality of the system's devices. The system may be configured for device-to-device communication. In an example, a first additional leadless device may (e.g., directly) sense the ventricular activity in the ventricle and send the sensory input to the leadless pacing device in the atrium. The sensory data is received and may be used for further processing by the leadless pacing device. This may be highly beneficial for the power threshold determination as outlined herein, since the sensory data in the near vicinity of the ventricle may be taken into account, which may mitigate the need for filtering interference signals, as might be the case in atrium based far-field sensing. The sensory data of the first additional sensor may only be taken into account at certain steps of the power threshold search (or the determination of ventricular activity and/or event corresponding to an atrial stimulation). As an example, after the test stimulation with a test stimulation power, as outlined above, the correspondingly sensed ventricular activity may be determined, additionally, or alternatively, based on sensory data from the first additional device. This may minimize falsely determined time window parameters since the signal would be based on the highly reliable signal directly from the ventricle. During further threshold search steps (e.g., after the time window parameters have been determined), the determination of an occurrence or absence may be based on the sensory input from the leadless device in the atrium. This may reduce computing complexity since the determination of the occurrence or absence may need less effort to be determined by the leadless pacing device itself and may not require a highly pure signal strength (e.g., since only a contraction has to be simply confirmed and it may not necessarily need to be measured in detail).

In an example, the leadless device may comprise a second additional sensor for directly sensing an atrial activity of the heart corresponding to the direct stimulation of the atrium. The second sensor may be configured for near-field sensing. This concept may enable the device to sense the atrially-evoked response signal and/or sense atrial events. The atrially-evoked response signal may be the direct response signal of the myocardium of the atrium to the atrial stimulus. The second additional sensor may share one or more parts with the sensor, the first additional sensor and/or with the stimulator or may have separate parts (e.g., one or more electrodes). It may, for example, comprise the same electrode as the sensor, and/or the stimulator. The second additional sensor may be constructed with a fractal coating, which may be a conductive material in an irregular manner deposited onto the sensor surface. This may increase the electrochemically active surface area, which may decrease a pacing polarization artifact and/or may increase the amplitude of the atrially-evoked response. Hence, this may enable the second additional sensor to pick up the atrially-evoked response signal without significant interference at the device-to-tissue interface and correctly determine the atrially-evoked response. This example pacing device capable of directly sensing atrial activity may also be comprised in the system as outlined herein. The stimulator and/or the sensor may also comprise the fractal coating without being specifically designed for near-field sensing as the second additional sensor.

It is noted that, while referred to as sensor and second additional sensor, both may be implemented within a single sensor unit (e.g., comprising a single electrode or set of electrodes). For example, the sensor unit may comprise an electrode or set of electrodes as described herein. From the electrode or set of electrodes, an electrical signal may be picked up that contains a P-wave signal (as the signal is picked up at the wall of the atrium) and an R-wave signal (far-field signal). For example, by means of one or more filters (e.g., time domain and/or frequency domain), R-wave and P-wave signals may be extracted and evaluated separately (e.g., by different algorithms and/or different electronic components of the sensor unit which thus implements first and second sensors). As such, the aspects described herein with respect to the sensor and the second additional sensor also apply to the respectively other sensor.

A third aspect relates to a method for determining a stimulation response carried out by a leadless pacing device implanted into an atrium of a heart. The method may comprise performing a direct stimulation of the atrium of the heart by the device at a predetermined stimulation power and/or a predetermined stimulation energy. Further it may comprise sensing a ventricular activity of the heart corresponding to the direct stimulation by the device. Further it may comprise determining an occurrence or an absence of a ventricular event corresponding to the direct stimulation.

In an example the method further comprises performing multiple or a plurality of direct stimulations with different stimulation powers and/or different stimulation energies to determine a threshold of the stimulation power and/or of the stimulation energy for which a ventricular event occurs. In other examples, one or more other parameters of a predetermined direct stimulation may be varied to determine a corresponding threshold.

A fourth aspect relates to a computer program which may comprise instructions to perform any of the methods described herein, when the instructions are executed by a computer. For example, the computer program may be stored on a leadless device, or a device in a system as described herein, which may comprise means to execute the computer program instructions. The computer program may allow an autarkic, automated implementation of the aspects described herein. Consequently, technical intervention from medical staff and the patient may be minimized.

While the above mainly related to sensing ventricular activity, this is not a mandatory part of the present disclosure. In an aspect, a leadless pacing device for implanting into an atrium of a heart may be provided that may comprise an implant anchor for connecting the device to an inner wall of the atrium, a stimulator for a direct stimulation of the atrium, and a sensor for directly sensing an atrial activity of the heart corresponding to the direct stimulation of the atrium. The leadless device may be further configured to implement the steps outlined herein mainly with respect to a sensed ventricular activity additionally, or alternatively, for the sensed atrial activity, e.g., to determine whether a direct stimulation leads to a corresponding atrial event, to determine a threshold, and/or to sense the atrial activity in a predetermined time window. Alternatively or additionally, the device may be configured to determine whether a direct stimulation at a predetermined stimulation power and/or a predetermined stimulation energy leads to a corresponding ventricular event.

Similarly, in an aspect, a method for determining a stimulation response carried out by a leadless pacing device implanted into an atrium of a heart may be provided. The method may comprise performing a direct stimulation of the atrium of the heart by the device at a predetermined stimulation power and/or a predetermined stimulation energy. Further it may comprise directly sensing an atrial activity of the heart corresponding to the direct stimulation by the device. Further it may comprise determining an occurrence or an absence of an atrial event corresponding to the direct stimulation.

It is noted that the method steps as described herein may include all aspects described herein, even if not expressly described as method steps but rather with reference to an apparatus (or device). Moreover, the devices as outlined herein may include means for implementing all aspects as outlined herein, even if these may rather be described in the context of method steps.

Whether described as method steps, computer program and/or means, the functions described herein may be implemented in hardware, software, firmware, and/or combinations thereof. If implemented in software/firmware, the functions may be stored on or transmitted as one or more instructions or code on a computer-readable medium. Computer-readable media include both computer storage media and communication media including any medium that facilitates transfer of a computer program from one place to another. A storage medium may be any available media that can be accessed by a general purpose or special purpose computer. By way of example, and not limitation, such computer-readable storage media can comprise RAM, ROM, EEPROM, FPGA, CD/DVD or other optical disk storage, magnetic disk storage or other magnetic storage devices, or any other medium that can be used to carry or store desired program code means in the form of instructions or data structures and that can be accessed by a general-purpose or special-purpose computer, or a general-purpose or special-purpose processor.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

FIG. 1 Schematic representation of an exemplary embodiment of a leadless pacing device and an optional system according to the present invention.

FIG. 2 Schematic representation of an exemplary embodiment of a method according to the present invention.

FIG. 3 Schematic representation of a time window that may be used by a method or a device according to the present invention.

### DETAILED DESCRIPTION

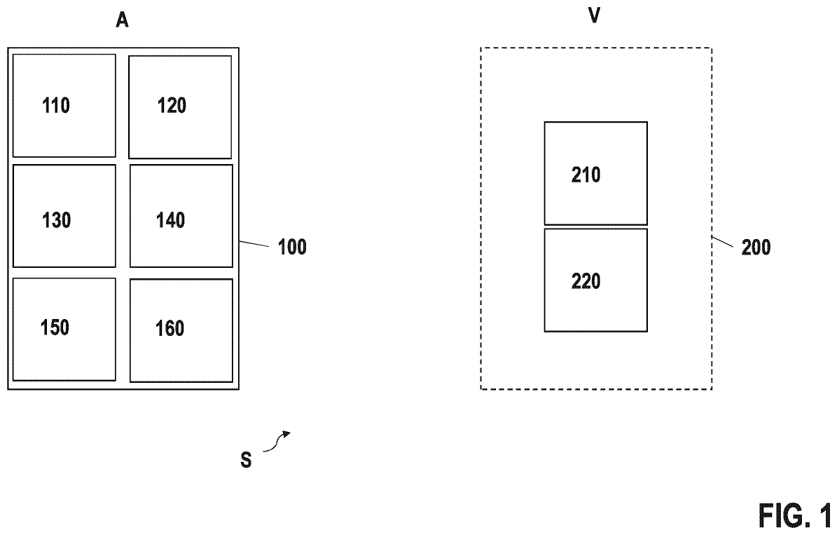

FIG. 1 shows a schematic of an exemplary leadless pacing device 100 according to the present invention.

The leadless pacing device 100 may optionally be part of a system S that may comprise a first additional sensor 200, wherein the first additional sensor 200 may be implantable in a ventricle V.

The leadless device 100 may be implanted into an atrium A of a heart (e.g., an atrium A of a human heart). The leadless device 100 may be particularly configured for implanting into a right atrium of a patient, for example.

25 The leadless pacing device 100 may comprise an implant anchor 110 for connecting the leadless device 100 mechanically to an inner wall of the atrium A. The implant anchor 110 may be constructed as the base mount of the leadless device 100, which may serve as a surgical fixation point during an implantation procedure. The implant anchor 110 may comprise one or more fixation tines and/or an anchor structure and/or a screw-in fixation 30 mechanism to enable a stable and reliable connection to the atrium A.

The leadless pacing device 100 may further comprise a stimulator 120. The stimulator 120 may be configured to apply electrical stimulation onto the inner wall of the atrium A. It may be configured to deliver a specific amount of electrical energy into the atrium A. The stimulator 120 may be connected to a power electronic circuitry which may provide defined energy for the stimulation (e.g., in the form of one or more stimulation bursts). The stimulation may be for testing, calibration, core brady and/or anti-tachycardia pacing purposes. The stimulator may comprise an electrically conductive material which may be formed by means of an electrode (e.g., a cathode or an anode), which may provide a direct electrical connection to the wall of the atrium A for transferring the electrical energy of the stimulator onto the surrounding tissue.

The amount of electrical energy applied by the stimulator 120 may be adaptable. For example, the electrical energy may be applied in the form of pulses with an adaptable energy and/or an adaptable pulse timing or frequency. Further adaptable parameters may be (average) power, cycle length, current, voltage, pulse width, FWHM, etc. of the applied electrical stimulus. The electrical energy and/or pulse parameters may be determined by the leadless device 100 or the system S (e.g., depending on sensed ventricular activity and/or atrial activity).

The leadless device 100 may further comprise a sensor 130 (stationed in the atrium A, being part of the leadless device 100). In an example, the sensor 130 may be configured to indirectly measure ventricular activity by sensing the signal from a ventricle V (e.g., right ventricle) of the heart (e.g., far-field sensing and/or accelerometer-based sensing). For far-field sensing, the sensor 130 may be configured to be in direct contact with the inner wall of the atrium A such that an electrical connection is established. Notably, the sensor 130 may be further configured to sense electrical signals present in the atrium A indicative of ventricular activity (e.g., the R-wave signal). The sensor 130 may comprise an electrically conductive material which may be formed by means of an electrode (e.g., a cathode or an anode) to directly pick up the respective electrical signal from the inner wall of the Atrium A. The sensor 130 or its electrode may comprise a coating (e.g., by galvanization), a texture (e.g., with a dimensioned porosity) and/or a specific geometry (e.g., screw shape, flat circular shape, etc.) which may be optimized with regards to the properties of the inner wall of the atrium A to ensure a well-functioning sensing contact. As an example, the ventricular activity may be determined to correspond to an atrial stimulus.

In an example, the sensor 130 and/or the stimulator 120 may share an element with the implant anchor 110, thus enabling the mechanical fixation and sensing/stimulating functions of the leadless device 100 by a single structural element, e.g., an electrode that at least in part also serves for mechanically connecting the leadless device 100 to the wall of the atrium A.

The leadless device 100 may further comprise a second additional sensor 150. The second additional sensor 150 may be in direct contact with the inner wall of the atrium A such that an electrical connection is established. The second additional sensor 150 may thus enable near-field sensing and may be configured to sense the atrial activity (e.g., P-wave signal). In an example, the sensor 150 may be configured to also sense and/or determine the atrially-evoked response signal (i.e., the direct response of the atrial myocardium to an atrial stimulus). The sensor 150 may comprise an electrically conductive material which may be formed by means of an electrode (e.g., a cathode or an anode) to directly pick up the respective electrical signal from the inner wall of the Atrium A. The second additional sensor 150 (e.g., in particular its electrode) may have a fractal coating on the surface which serves as the contact to the surrounding (atrial) tissue. The fractal coating may be of a conductive material (e.g., iridium, titanium nitride) which may be deposited to form an irregular shape at a microscopic scale. The coating may thus significantly increase the electrochemically active surface area, which is beneficial to determine the atrially-evoked response. Hence, this may enable the second additional sensor to pick up the atrially-evoked response signal without significant interference at the device-to-tissue interface and correctly determine the atrially-evoked response. For example, the fractal coating may ensure that polarization artifacts remain consistent, which can then be accurately considered in the further signal processing. This may reduce or completely inhibit the detection of false positives and/or false negative. The atrially-evoked response may be determined to correspond to an atrial stimulus, for example.

The stimulator 120, the sensor 130 and/or the second additional sensor 150 may share one or more electrodes and/or have separate electrodes. The stimulator 120 and/or the sensor 130 may also have a fractal coating as described with reference to the second additional sensor 150 when their electrodes are shared with the second additional sensor or when their respective electrodes are separate.

As outlined above, in some examples, the functionalities of the sensor 130 and the second additional sensor 150 may be provided by a (single) sensor unit of the leadless device 100. In some examples, leadless device 100 may be provided with a sensor unit implementing the function of second sensor 150 and/or sensor 130, instead of second additional sensor 150 and sensor 130.

The leadless device 100 may further comprise a battery 140, which may serve as power supply of the leadless device 100. The battery 140 may have a slightly smaller form factor compared to batteries used for devices that are implanted into a ventricle, due to the smaller size of the atrium A.

Further, the leadless device may comprise a control unit 160. The control unit 160 may be at least one computing unit (e.g., a microprocessor, a microcontroller, an embedded system, an electronic circuitry, etc.) which may implement computing instructions. It may control various device elements based on a configuration, which may be defined by the computing instructions (e.g., by a computer program running on the control unit 160). The control unit 160 may be connected to the various device elements outlined above (e.g., over one or more input/output ports for respective electrical signaling) to control and/or receiving/send information. The control unit 160 may, for example, receive sensory input from the sensors outlined herein, and apply further signal processing (e.g., scaling, filtering, rectification). The control unit 160 may have its own memory and/or may be coupled to a separate memory which may be comprised in the leadless device 100.

The control 160 unit as described herein may also be implemented in hardware, software, firmware, and/or combinations thereof, for example, by means of one or more general-purpose or special-purpose processors and/or microcontrollers.

The control unit 160 may be coupled to the stimulator 120 and/or a power electronic circuitry of the stimulator 120, wherein a specific signaling of the control unit 160 may result in a desired stimulation output with a set of stimulation parameters over the stimulator 120 onto the atrium A of the heart. The control unit 160 may thus control the stimulation output in the atrium A, which may be based on several input factors, which may be processed and analyzed by the control unit 160. As an example, the paced output may be a response to a ventricular activity and/or atrial activity corresponding to a prior atrial stimulation. The control unit 160 may thus enable various implementations of cardiac therapies by the device.

As an example, the leadless pacing device 100 may be configured to support AAI mode cardiac therapies for patients with sick sinus.

It is noted that the way in which the interaction between control unit 160 and stimulator 120, and sensors 130 and 150 was described is merely optional. At least in part, functions of control unit 160 may be implemented by the sensors themselves. For example, the second additional sensor 150 may, in some examples, share an electrode with the sensor 130, such that their signals may physically be picked up by the same elements. However, they may comprise different signal processing/filtering etc. to derive atrial and ventricular activity, respectively, implemented by one or more control units.

The system S may optionally comprise a first additional sensor 200 according to the present invention which is indicated by dashed lines in FIG. 1. The first additional sensor 200 may be implanted into a ventricle V of a heart (e.g., a ventricle V of a human heart). The first additional sensor 200 may particularly be configured for implanting into a right ventricle of a patient. In an example, the first additional sensor 200 may be a passive sensor that detects the ventricular activity. The first additional sensor 200 may comprise a capacitor 210 as a passive sensor which may be implanted into the ventricle V in such a way that it is coupled to the ventricular activity. The capacitor 210 may be aligned in the tissue, so that the ventricular activity of the nearby muscle cells may influence the electrical properties of the capacitor 210. For example, the capacitor 210 may comprise a dielectric medium interposed between to conductors. The first additional sensor 200 may be arranged such that electrical fields of the ventricular tissue depolarizations influence the dielectric medium leading to a change of the dielectric medium, resulting in a change in the capacitance of the capacitor 210. In another example, the mechanical contractions of the ventricular cells may influence the mechanical properties of the capacitor 210 (e.g., the distance between capacitor plates and/or the area of the capacitor plates), which may lead to a change in capacitance of the capacitor 210. The capacitor 210 may be coupled to an inductor (not shown) to form a resonant circuit whose resonant frequency would then depend on the capacitance of the capacitor 210 and the inductance of the inductor. The inductance of the inductor may be designed to have a fixed value, wherein the inductor may be configured to not significantly change its inductance under the direct influence of the ventricular contractions and/or excitations. The resonant frequency of the resonant circuit may therefore be significantly dependent on the value of the capacitance of the capacitor 210 (and not the inductance) under the ventricular activity. A readout of the resonant frequency corresponding to a change in capacitance of the capacitor 210 may thus enable a readout of the ventricular activity. In an example, the resonant circuit in the ventricle V may comprise an antenna 220 which is connected to the inductor. The antenna 220 may relay the information of the resonant circuit (e.g., resonant frequency, the change in resonant frequency, the change in capacitance and/or the capacitance itself) to the leadless device 100 stationed in the atrium A. The leadless device 100 may comprise a receiver unit to receive the information. The receiver unit may be coupled to the control unit 160, which may further process the signal. In another example. an antenna 220 may be comprised in the leadless device 100 stationed in the atrium whereas the antenna 220 is configured to be coupled to the resonant circuit and to read out the resonant circuit parameters as outlined above. The first additional sensor 200 may comprise a power source to ensure application. In another example the first additional sensor 200 may be configured to not require an internal power source for detecting the ventricular activity.

In an example, the first additional sensor 200 may be comprised by a ventricularly-stationed leadless pacing device, which may be configured to directly sense the ventricular activity. The ventricularly-stationed leadless pacing device may comprise device elements that may be similar to those described herein for the leadless device 100 in the atrium. Its device elements may have features configured for the ventricle V. The optional ventricular leadless pacing device and the leadless pacing device 100 may be configured for device-to-device communication, such that the sensed activity may be requested, as well as exchanged between the devices in a manner similar to the relay of pacing commands.

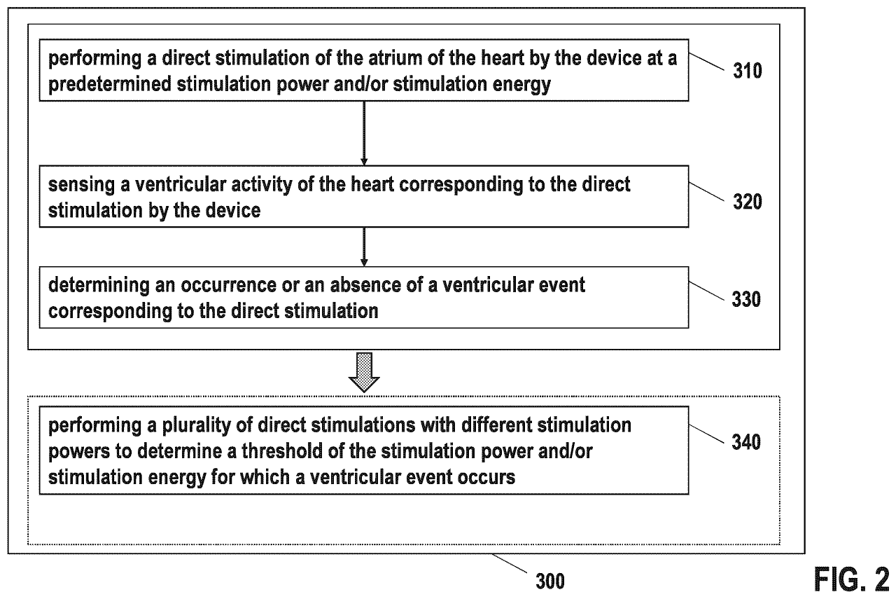

FIG. 2 shows a schematic of an exemplary method 300 according to the present invention for determining a stimulation response carried out by a leadless pacing device implanted into an atrium of a heart. The method may be performed by the leadless device 100 implanted into an atrium A of a heart (e.g., by the control unit 160 of the leadless device 100). The method may be also performed by the leadless device 100 in the system S configuration as outlined herein.

The method 300 may comprise performing 310 a direct stimulation of the atrium of the heart by the leadless device 100 at a predetermined stimulation power and/or a predetermined stimulation energy. The direct stimulation may be based on a stimulation parameter or a set of stimulation parameters (e.g., cycle length, current, voltage, pulse width, total power, average power, total energy, etc.) corresponding to an electrical power and/or electrical energy of the stimulation. For example, the direct stimulation may comprise a fixed pulse duration, wherein the pulse amplitude may be adjusted to adapt the stimulation power. Notably, the stimulation power and/or stimulation energy (and/or the stimulation parameters) may be set by the control unit 160. The control unit 160 may activate the stimulation, which may be performed by the stimulator 120.

Subsequently, the method 300 may comprise sensing 320 a ventricular activity of the heart corresponding to the direct stimulation of the device. The ventricular activity as a heart response may be medically associated with the corresponding atrial stimulation. For example, the atrial stimulation may evoke an atrial signal which may be conducted along the heart to the ventricle to cause a corresponding ventricular activity as a heart response (e.g., a ventricular contraction). Additionally or alternatively, the method may comprise sensing an atrial activity (e.g., an atrial event and/or an atrially-evoked response signal) of the heart corresponding to the direct stimulation of the device. For example, as stated above, the atrial stimulation may evoke an atrial signal as a direct heart response which may be sensed, as well.

The ventricular activity may be sensed by the sensor 130 and/or the first additional sensor 200, whereas the atrial activity may be sensed by the second additional sensor 150. The sensory data may be transferred to the control unit 160, which may apply initial signal processing (e.g., high-gain signal amplification). The control unit 160 may be further configured to control the resolution of the sensory data, which may be implemented by adapting sensor settings, and/or by postprocessing of the raw sensor data. This may enable the leadless device 100 to collect high-resolution data, which may also comprise a high-gain signal of an intracardiac electrogram (IEGM). This processing may ensure a reliable signal quality for further processing steps.

As outlined herein, sensor 130 and/or second additional sensor 150 may be implemented as a (single) sensor unit (e.g., with a different channel for the far-field channel and/or for the near-field signal, respectively), such as to implement the two different sensor functionalities.

Subsequently, the method 300 may comprise determining 330 an occurrence or an absence of a ventricular event corresponding to the direct stimulation. The corresponding ventricular event may be a ventricular contraction taking place in response to the applied atrial stimulation. Additionally, or alternatively, the method 300 may comprise determining an occurrence or an absence of an atrial event and/or an atrially-evoked response corresponding to the direct stimulation. The atrial event may be an atrial contraction, whereas the atrially-evoked response may be a particular signature of the atrially-evoked response signal. The determination of a ventricular event, atrial event and/or atrially-evoked response may be implemented by a respective detection algorithm which may be implemented by the control unit 160. The respective detection algorithm may require signal processing (e.g., scaling, filtering, rectification, signal amplification, etc.).

The method 300 may further comprise performing 340 multiple direct stimulations (of the atrium A) with different stimulation powers and/or stimulation energies to determine a threshold of the stimulation power and/or stimulation energy for which a ventricular event (and/or atrial event and/or atrially-evoked response) occurs. This may enable the determination of a minimum stimulation power and/or a minimum stimulation energy which may still result in the occurrence of a ventricular event (and/or atrial event and/or atrially-evoked response). The method step of performing 340 may be implemented by sequentially repeating the steps of performing 310, sensing 320, determining 330, wherein each sequence may be based on a stimulation with a different stimulation power and/or stimulation energy, which may be adjusted after each sequence as outlined herein. The sequential stimulation may also be referred to as search stimulations.

Hence, the method 300 may enable various types of atrial capture threshold searches for various heart responses (e.g., ventricular event, atrial event, atrially-evoked response) to an atrial stimulus. A successful atrial capture may be understood as an occurrence of a specific heart response to the atrial stimulation (i.e., capture confirmation). An unsuccessful atrial capture may be understood as the absence of a specific heart response to the atrial stimulation (i.e., capture loss).

The atrial capture threshold search may search for a value of an atrial stimulation power and/or stimulation energy (and/or any other stimulation parameter) which marks a minimum threshold for effecting the specific heart response. The specific heart response may not be significantly dependent on the amount of a stimulation parameter of the stimulation, but merely dependent on the stimulation parameter being above or below its specific threshold. For example, the applicable stimulation power of the leadless device may vary in a certain range. The minimum threshold of a heart response (which may be patient specific) may, for example, be at 30% of that range. Stimulations with power levels above or equal to 30% may result in the specific heart response, stimulations with power levels below 30% may not cause the specific heart response.

For brevity purposes, the atrial capture threshold search will be explained in further detail in an example for a ventricular contraction as the specific heart response to an atrial stimulus. However, this is just for exemplary purposes and, in other examples, the method 300 may be based on a different heart response (i.e., a different ventricular signal, an atrial event and/or an atrially-evoked response).

To determine the threshold, the leadless pacing device 100 may evaluate a sequence of search stimulations (i.e., atrial search paces) and determine the respectively conducted ventricular activity, as outlined for the method 300. The steps of sensing 320 and determining 330 of the ventricular activity may be narrowed to a time window after the search stimulation.

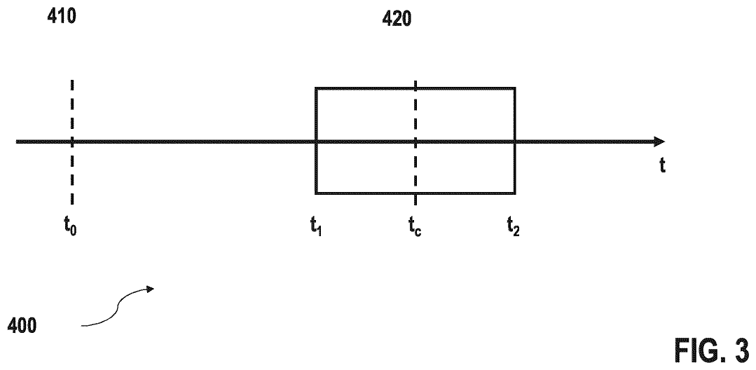

An exemplary time window 420 is shown in the exemplary time diagram 400 of FIG. 3. Notably, when (e.g., in method 300), a time window 420 is used, ventricular activity outside of the time window 420 may not be sensed and/or taken into account for the step of determining 340. Only the signal in the time window 420 may be deemed relevant. This may significantly reduce computing complexity for the control unit 160 and may save power consumption leading to an increased device longevity. The time window 420 may be associated with an expected Vs (ventricle sensed) window assumed for a typical AV delay of the heart. The AV delay may be related to the signal conduction from the atrium to the ventricle. It may be seen as a time when the ventricular contraction will normally occur after an atrial stimulation. During the atrial capture threshold search, a steady AV delay and/or a stable ApVs (atrial paced, ventricle sensed) rhythm may be achieved by altering the atrial stimulation rate, which may, for example, be increased to overdrive the intrinsic heart activity. This may achieve a stable one-to-one AV conduction. Furthermore, an AV pacing delay may be lengthened to encourage sensing. Overall, atrial stimulation parameters may be adapted during the capture threshold search to reliably achieve that the response of ventricular activity is reliably taking place in the time window 420 after the atrial stimulus. This may enable to narrow the confirmation of a capture or capture loss to the time window 420 after a search stimulation 410 was applied.

The time window 420 is shown in FIG. 3 on a time scale relative to the search stimulation 410. The atrial search stimulation 410 may occur at a time to. The time window may be set to be in a period when the ventricular response is expected if a successful capture occurs, as outlined herein. The time window 420 may have a time window length defined by two time values which mark the beginning time t₁ and the end time t₂ of the time window 420. The time t₁ and t₂ may be referred back to the time to, which may be defined as the initial reference time to =0, when the atrial stimulation occurs. For example, a time measurement may be started after applying the atrial stimulation (e.g., by means of a time counter). The time may be measured by the control unit 160, and/or it may be measured by an additional time unit comprised in the leadless device 100. An important parameter of the time window 420 may be the center time t_{C} which can be defined as the time window center, which may be calculated as t_{C}=(t₂+t₁)/2. The center time t_{C} may be set to be in the amplitude peak (or center of the distribution) of the expected ventricular contraction signal. The time window length (t₂−t₁) may be set to take into account cycle-to-cycle variations of the ventricular contractions. This may ensure that the signal associated with the occurrence of a ventricular contraction may still be determined inside the time window 420, even when the signal of a ventricular contraction is slightly shifted.

The parameters of the time window 420 may be based on the patient's ApVs interval history. The center time t_{C} may be set to match the last saved ApVs interval, or an average of a short buffer history of ApVs intervals.

The capture search may comprise an initialization phase to determine parameters of the time window (e.g., t_{C}, t₁, t₂, window length etc.) by a continuous measurement of ventricular activity after a test stimulation. The test stimulation may comprise a parameter (e.g., a high-power pulse) or a set of parameters which are expected to successfully capture a ventricular contraction. Subsequently, the control unit 160 may analyze the signal of the continuous measurement of the ventricular activity and determine at least one time window parameter, as outlined herein. In an example, the test stimulation may be done to determine only the center time t_{C}, wherein the time window length may be based on a predetermined time window length, which may be defined by the clinician and/or technician (i.e., by an external configuration of the leadless device 100). During the initialization phase, the atrial stimulation parameters (e.g., atrial stimulation rate) may be adapted for the same conditions as during the threshold search to achieve stable conduction conditions, to ensure that the determined time window parameters are the same as during the capture threshold search. This would imply that several test stimulations would be performed. The continuous measurement could then be performed after one of the test stimulations or a subset thereof, or possibly after each one of the test stimulations.

Coming back to FIG. 2, particularly in the step 340 the substep(s) of sensing and/or determining may thus be narrowed to the time window 420 without the loss of information if a successful capture occurred or not. The threshold search may be based on progressively sweeping a parameter (or a set of parameters) of the stimulation and determining if the respective stimulation results in a capture confirmation or capture loss inside the time window 420. As outlined herein, the threshold search may start with a high-power stimulation expected to capture, with a subsequent incremental decrease of the power of the stimulations to reach the vicinity of the threshold to eventually determine the latter. Various other threshold search algorithms may be applied.

As an example, the threshold search may be optimized to require as few steps as possible, which may be achieved by starting the threshold search based on a stored threshold. The stored threshold may be the last saved threshold, or an average of a short buffer history of prior thresholds. In a further example, the time window parameters may be determined after each threshold search step or may be determined at certain points (e.g., after certain steps) in the threshold search. For example, after a successful capture threshold determination, the time window parameters may be determined again as confirmation, to ensure they have not drifted during the search.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. Leadless pacing device for implanting into an atrium of a heart, comprising:
an implant anchor for connecting the device to an inner wall of the atrium;
a stimulator for a direct stimulation of the atrium;
a sensor for sensing a ventricular activity of the heart corresponding to the direct stimulation of the atrium.

2. Leadless pacing device according to claim 1, wherein the device is further configured to determine an occurrence or an absence of a ventricular event corresponding to the direct stimulation of the atrium, based at least in part on the sensed ventricular activity.

3. Leadless pacing device according to claim 1, wherein the device is configured to determine whether a direct stimulation at a predetermined stimulation power and/or a predetermined stimulation energy leads to a corresponding ventricular event.

4. Leadless pacing device according to claim 1, wherein the device is further configured to change a stimulation power and/or a stimulation energy of the direct stimulation.

5. Leadless pacing device according to claim 3, wherein the device is further configured to perform multiple direct stimulations with different stimulation powers and/or different stimulation energies to determine a threshold of the stimulation power and/or of the stimulation energy for which a ventricular event occurs.

6. Leadless pacing device according to claim 1, wherein the device is further configured to sense the ventricular activity and/or to determine the ventricular event in a predetermined time window after the direct stimulation.

7. Leadless pacing device according to claim 6, wherein the device is further configured to determine at least one parameter of the time window at least in part based on data, stored by the device, concerning at least one previously measured time interval of the heart.

8. Leadless pacing device according to claim 6, wherein the device is further configured to determine at least one parameter of the time window at least in part based on performing a test stimulation with a test stimulation power and/or with a test stimulation energy and sensing the corresponding ventricular activity.

9. Leadless pacing device according to claim 1, wherein the sensor is configured for far-field sensing.

10. Leadless pacing device according to claim 1, wherein the sensor is configured for receiving information on ventricular activity from at least one first additional sensor for implanting in a ventricle of the heart.

11. System comprising the leadless pacing device and the at least one first additional sensor according to claim 10.

12. Leadless pacing device according to claim 1, comprising a second additional sensor for directly sensing an atrial activity of the heart corresponding to the direct stimulation of the atrium.

13. Method for determining a stimulation response carried out by a leadless pacing device implanted into an atrium of a heart, comprising:
performing a direct stimulation of the atrium of the heart by the device at a predetermined stimulation power and/or a predetermined stimulation energy;
sensing a ventricular activity of the heart corresponding to the direct stimulation by the device;
determining an occurrence or an absence of a ventricular event corresponding to the direct stimulation.

14. Method according to claim 13, further comprising:
performing multiple direct stimulations with different stimulation powers and/or
different stimulation energies to determine a threshold of the stimulation power and/or of the stimulation energy for which a ventricular event occurs.

15. Computer program comprising instructions to perform a method of one of claim 13, when the instructions are executed by a computer.
